(19) United States
(12) Patent Application Publication
(10) Pub. No.: US 2022/0087839 A1
(43) Pub. Date: Mar. 24, 2022

(54) Arterial Drug Eluting Device to Treat Diabetes With Targeted Delivery of Medication

(71) Applicant: Becton, Dickinson and Company, Franklin Lakes, NJ (US)

(72) Inventors: Murtaza Amin, Farmington, UT (US); Brendan Laine, Salt Lake City, UT (US)

(21) Appl. No.: 17/475,616

(22) Filed: Sep. 15, 2021

Related U.S. Application Data
(60) Provisional application No. 63/080,077, filed on Sep. 18, 2020.

(51) Int. Cl. A61F 2/90; A61M 5/142; A61M 5/172; A61L 31/16

(52) U.S. Cl. CPC A61F 2/90; A61M 5/14276; A61F 2250/0067; A61L 31/16; A61M 2230/201; A61M 5/1723

(57) ABSTRACT

Disclosed are methods for treating diabetes comprising methods and devices for reducing the levels of glucagon found in diabetic patients. Disclosed is a stent comprising a biocompatible polymer containing at least one glucagon suppressing drug, the stent is inserted into an artery or vein supplying the pancreas and as the drug is eluted it reduces the level of glucagon. In another embodiment, the method of treating diabetes comprises providing a pump having a catheter and a reservoir containing at least one glucagon suppressing drug, inserting the catheter into an artery supplying blood to the pancreas, and infusing the glucagon suppressing drug into the arterial supply.

This application claims priority to U.S. PROVISIONAL Patent Application Ser. No. 63/080,077, filed Sep. 18, 2020, the disclosure of which is incorporated herein by reference in its entirety.

### TECHNICAL FIELD

This present disclosure relates generally to methods of treating diabetes, and more particularly to a device and system for treatment of diabetes by targeted delivery of medications.

### BACKGROUND

This section provides general background information which is not necessarily prior art to the inventive concepts associated with the present disclosure.

The present disclosure is directed to methods for treating diabetes. The disease of diabetes at its most basic level involves a dysfunction in the body's ability to regulate blood glucose levels. Blood glucose levels in a person without diabetes are maintained within a “normal range” by the hormone insulin which functions to lower blood glucose levels as they rise, for example, following a meal and by glucagon which functions to raise blood glucose levels as they fall below the normal range, for example due to activity or time since a last meal. In a non-disease state these two hormones fluctuate, having opposite actions on blood glucose levels, to maintain blood glucose levels within the normal range. Insulin is produced by beta cells in islet of Langerhans cells in the pancreas while glucagon is produced by alpha cells in the islets of Langerhans cells in the pancreas. The pancreas serves both an exocrine function and an endocrine function in the body. Approximately 95% of the cells in the pancreas are exocrine cells and they produce, among other things, the digestive enzymes that are moved from the pancreas through a system of ducts into the small intestine at the point where it joins to the stomach. The other 5% of the cells in the pancreas are the islets of Langerhans cells, which comprise the alpha, beta and delta cells and which, respectively, secrete glucagon, insulin and somatostatin. Insulin acts like a “key” to unlock cells in the body allowing entry of glucose into the cells for fuel. Any excess blood glucose is typically converted by the liver into glycogen which is stored in the liver and in skeletal muscles. When blood glucose levels fall below the normal range glucagon is released from the pancreas alpha cells and this glucagon functions to cause conversion of stored glycogen back into glucose for use by cells as fuel. In this way blood glucose is maintained within a normal range. Glucagon also promotes gluconeogenesis to form glucose from 3-carbon substrates including amino acids, lactate and glycerol and by lipolysis to break down stored triglycerides into fatty acids for fuel usage by cells. The brain is unique in that it requires glucose for fuel as the neurons cannot effectively use either amino acids or fatty acids as a fuel source, thus glucose is essential for brain functionality.

People with diabetes are categorized as having either type 1 or type 2 diabetes. There are approximately 10 million people worldwide who have type 1 diabetes and approximately 400 million people worldwide with type 2 diabetes. Type 1 diabetes is characterized by a patient that produces very little to no insulin. Although the exact causes of type 1 diabetes are not known, in most cases it appears that the body's immune system attacks the insulin-secreting beta cells of the pancreas. The attacked beta cells die or lose their function leading to a lack of insulin. Other potential causes of type 1 diabetes include genetics, viral infections or environmental causes that damage the beta cells. In all cases the large reduction in or complete lack of insulin leads to continuously elevated blood glucose levels, increased catabolism and sarcopenia. If untreated, this lack of insulin and thus lack of blood glucose control results in ketoacidosis and death.

Type 2 diabetes is typified by an insensitivity to the action of insulin, known as insulin resistance, coupled with an inability to secrete enough insulin to prevent an excess secretion of glucagon from the alpha cells, through a lack of a local within the pancreas insulin effect to control the secretion of glucagon from the alpha cells. The excess glucagon also contributes to the drive for raised blood glucose. In addition, other cells in the body also become less sensitive to the effects of insulin and as a result glucose does not move out of the blood and into the cells as effectively. As type 2 diabetes progresses the loss of beta cell function also progresses, rendering a state of increasing insulin deficiency.

Current treatment of type 1 diabetes requires injecting a quick acting insulin sub-dermally with meals and this is often paired with a once a day injection of a longer acting insulin. It also requires that the patient frequently check their blood glucose levels using a blood glucose meter, certainly before each meal and usually before bedtime. The mealtime dose of insulin is dependent upon the measured blood glucose level in combination with the amount of carbohydrates and glycemic effect of the food eaten. Essentially, the diabetic patient needs to accurately determine the amount of carbohydrates in the meal and then dose a certain amount of insulin based on the carbohydrate ratio prescribed by their doctor and taking into account any adjustment factor up or down to the calculated dose based on the measured blood glucose. The carbohydrate ratio refers to the pre-determined ratio of units of insulin to inject per gram of carbohydrate taken in the meal. This ratio is determined in conjunction with the patient's endocrinologist and often needs to be adjusted as the disease progresses or if the patient begins a more rigorous exercise program or increased activity level. The reason is that exercise and activity can increase a person's sensitivity to insulin, a good thing for a diabetic, meaning less insulin is needed to achieve reduction of circulating glucose. Newer therapy methods include use of an insulin pump connected to a subdermal cannula that is replaced approximately every three days. The insulin pump has a replaceable reservoir containing a quick acting insulin such as Humalog. Working with the endocrinologist the patient has the pump set up to deliver a continuous basal rate of insulin, which can be varied over the 24 hours of a day. The pump is also setup to deliver a bolus of insulin at mealtimes as directed by patient input. Prior to a meal the patient checks their blood glucose level and determines the amount of carbohydrates in the meal. Then this data is input into the pump and a dose of insulin is delivered by the pump based on the pre-set carbohydrate ratio with any corrections for the measured blood glucose level.

The pump system has recently been supplemented by including a continuous glucose monitoring sensor that communicates with the insulin pump. The continuous glucose monitor sensors comprise a glucose measuring probe that is inserted sub-dermally and a transmitter that is connected to the probe and taped to the surface of the body. The glucose measuring probe needs to be replaced generally on a weekly basis. The probe measures the interstitial glucose levels, generally every 5 to 10 minutes and this data is sent to the transmitter and the transmitter transmits the data to the pump. The pump includes software and uses an algorithm to adjust the basal rate of insulin based on the accumulated data and blood glucose trends. Thus, the system is a pseudo pancreatic loop feedback system using only insulin. In all of these methods of treating type 1 diabetes the amount of insulin that the patient must inject to control their blood sugar levels is always much higher than the body would normally release in response to the same blood glucose levels. Because the insulin is injected sub-dermally rather than being released from beta cells directly in the blood stream there are timing and absorption issues. In a non-diabetic person insulin secreted from the beta cells enters the portal circulation which goes from the pancreas to the liver first before entering the systemic circulation. In other words, in a non-diabetic person insulin has a preferential effect at the liver first before acting in the rest of the body and the liver sees much higher levels of insulin than the rest of the body. This is very different from the pharmacologically delivered insulin in a person with diabetes, which enters the systemic circulation from its sub-dermally delivered insulin depot so that all body tissues see the same amount of insulin. This is a key difference that explains some of the obstacles in pharmacological sub-dermally delivered insulin being able to replicate the normal physiological actions of insulin. That being said, the use of an insulin pump and continuous glucose monitoring system is the most optimized therapeutic means to replicate the physiological actions of insulin, although issues still remain.

In type 2 diabetics, many times insulin levels may be elevated, especially early on in the progression of the disease and this elevation causes additional health problems. Treatment for type 2 diabetics often begins with dietary changes to reduce carbohydrate intake and to reduce total caloric intake. Often a type 2 diabetic benefits from weight loss. If these changes are not sufficient to reduce blood glucose levels then the second stage often adds in oral medications to attempt to reduce blood glucose levels. These medications are not insulin instead they act on different aspects of blood glucose control. These include, by way of example: alpha-glucosidase inhibitors which aid in breakdown of starches; biguanides which decrease how much sugar the liver makes, decrease intestinal sugar absorption, crease insulin sensitivity and helps muscles to absorb more glucose; dopamine agonists which may affect body rhythms and prevent insulin resistance; dipeptidyl peptidase-4 inhibitors which help raise levels of the insulinotropic hormone, glucagon-like peptide-1; glucagon-like peptide-1 receptor agonists which stimulate beta cell secretion of insulin and suppresses alpha cell secretion of glucagon; meglitinides which help the body to release insulin; sodium-glucose transporter 2 inhibitors which work by preventing the kidneys from holding on to glucose, thereby allowing for loss of glucose in the urine; sulfonylureas which stimulate the insulin secretion from the beta cells; and thiazolidinediones which work by decreasing insulin resistance and allowing natural endogenous insulin to work more effectively. Eventually, if these other medications do not work alone or in combination to control blood glucose levels the type 2 diabetic may need to begin treatment with insulin like a type 1 diabetic.

As discussed in any treatment of diabetes with insulin the levels of insulin required to be injected to control blood glucose levels are always much higher than what the body of a non-diabetic sees because it is injected sub-dermally. Continuously elevated insulin, which results from these forms of treatment: promotes lipogenesis while inhibiting lipolysis leading to an accumulation of adipose tissue, especially at insulin injection sites; promotes cellular proliferation and increasing the risk of some cancers; inhibits apoptosis; promotes hypertension; and promotes vascular plaque formation. Complications from diabetes often include hypertension and a range of cardiovascular health problems. Too high of a dose of insulin can cause a rapid drop in blood sugar, hypoglycemia, which can be acutely life-threatening. In general, the higher the dose of insulin required, the more volatile the blood glucose levels are. Blood sugar volatility is dangerous in and of itself as high spikes cause long term health consequences, while rapid falls in blood glucose levels can be acutely life-threatening, leading to coma and death.

All treatments to date for diabetes, especially for type 1 diabetics, revolve around supplementation with insulin or effects on insulin usage by the body. As discussed above, however, excess circulating levels of insulin required by these treatment options brings about another set of health issues that are best avoided. Recent data suggests that more attention should be paid to the other side of the blood glucose equation, namely control of glucagon in a diabetic patient. Thus, it would be beneficial to develop treatment options that reduce the amount of insulin required to treat diabetes and thereby reduce the other health effects caused by elevated insulin levels.

### SUMMARY OF THE INVENTION

This section provides a general summary of the present disclosure and is not intended to be interpreted as a comprehensive disclosure of its full scope or all features, aspects and objectives.

An object of the disclosure is to provide an effective device for eluting a drug for the treatment of diabetes.

According to a first aspect of the disclosure, a stent is provided as a treatment for diabetes. The stent comprises a metal mesh scaffolding as well as a biocompatible polymer coating. The biocompatible polymer coating may contain at least one glucagon suppressing drug. The drub may elute from the biocompatible polymer over time.

In one disclosed embodiment, the metal mesh scaffolding may comprise chromium in combination with cobalt, platinum or a combination thereof. The biocompatible polymer coating of the scaffolding may comprise, for example, any of poly(L-lactic acid), a polymer comprising one or more amino acids, poly(lactic-co-glycolic acid), polycaprolactone, poly(vinylidene fluoride-co-hexafluoropropylene), a poly(ethylene glycol) poly(L-alanine-co-L-phenyl alanine) co-polymer, block co-polymers of poly(ethylene glycol) and poly(caprolactone), or combinations thereof.

The at least one glucagon suppressing drug may comprise any of somatostatin, a somatostatin analogue, leptin, a leptin analogue, amylin, an amylin analogue, insulin, and insulin analogue, or combinations thereof. In one example, the at least one glucagon suppressing drug may elute from the biocompatible polymer coating at a rate of from 50 to 500 milligrams per year.

Another aspect of the disclosure relates to a method of treating diabetes. The method may include the step of providing a stent comprising a metal mesh scaffolding, stent comprising a biocompatible polymer coating and the biocompatible polymer coating containing at least one glucagon suppressing drug wherein the drug can elute from the biocompatible polymer coating over time. In addition, the method may include the step of identifying a patient having diabetes. The method may further include inserting the stent into an artery or a vein supplying blood to the pancreas of the identified patient, thereby treating the diabetes.

In one embodiment, the provided metal mesh scaffolding may comprise chromium in combination with cobalt, platinum or a combination thereof. In this or other examples, the biocompatible polymer coating may comprise poly(L-lactic acid), a polymer comprising one or more amino acids, poly(lactic-co-glycolic acid), polycaprolactone, poly(vinylidene fluoride-co-hexafluoropropylene), a poly(ethylene glycol) poly(L-alanine-co-L-phenyl alanine) co-polymer, block co-polymers of poly(ethylene glycol) and poly(caprolactone), or combinations thereof.

In this embodiment or in other embodiments, the at least one glucagon suppressing drug of the biocompatible polymer coating may comprise somatostatin, a somatostatin analogue, leptin, a leptin analogue, amylin, an amylin analogue, insulin, and insulin analogue, or combinations thereof. The at least one glucagon suppressing drug may elute from the biocompatible polymer coating at a rate of from 50 to 500 milligrams per year.

In any of the above embodiments or in any other embodiments, the inserting step may comprise inserting the stent into one of the celiac artery, the superior mesenteric artery, the inferior mesenteric artery, the splenic artery, the superior pancreaticoduodenal artery, the inferior pancreaticoduodenal artery, or a vein supplying blood to the pancreas.

A third aspect of the disclosure relates to another method of treating diabetes. This method may include providing a pump having a catheter and at least one reservoir containing at least one glucagon suppressing drug. The method may further comprise identifying a patient having diabetes and inserting the catheter into an artery supplying blood to the pancreas of the identified patient. In addition, the method may include infusing the at least one glucagon suppressing drug into the artery from the catheter, thereby treating the diabetes.

In one embodiment, the at least one glucagon suppressing drug may comprise any of somatostatin, a somatostatin analogue, leptin, a leptin analogue, amylin, an amylin analogue, insulin, and insulin analogue, or combinations thereof.

In the above embodiment or in other embodiments, the inserting step may comprise inserting the catheter into one of the celiac artery, the superior mesenteric artery, the inferior mesenteric artery, the splenic artery, the superior pancreaticoduodenal artery, or the inferior pancreaticoduodenal artery.

The method may further comprise the step of providing a continuous glucose monitor sensor, wherein the continuous glucose monitor sensor may measure interstitial glucose levels and communicating the same to the pump. The pump may adjust a rate of infusion of the at least one glucagon suppressing drug based on the measured interstitial glucose level.

In these or other embodiments, the method may include the further step of implanting the pump into the identified patient.

These and other features and advantages of this disclosure will become more apparent to those skilled in the art from the detailed description herein. The drawings that accompany the detailed description are described below.

### BRIEF DESCRIPTION OF THE DRAWINGS

The drawings described herein are for illustrative purposes only of selected aspects and not all implementations, and are not intended to limit the present disclosure to only that actually shown. With this in mind, various features and advantages of example aspects of the present disclosure will become apparent to one possessing ordinary skill in the art from the following written description and appended claims when considered in combination with the appended drawings, in which:

FIG. 1 shows a schematic diagram of a drug eluting device according to a first embodiment; and

FIG. 2 shows a schematic diagram of a drug eluting device according to a second embodiment.

### DETAILED DESCRIPTION

In the following description, details are set forth to provide an understanding of the present disclosure.

For clarity purposes, example aspects are discussed herein to convey the scope of the disclosure to those skilled in the relevant art. Numerous specific details are set forth such as examples of specific components, devices, and methods, in order to provide a thorough understanding of various aspects of the present disclosure. It will be apparent to those skilled in the art that specific details need not be discussed herein, such as well-known processes, well-known device structures, and well-known technologies, as they are already well understood by those skilled in the art, and that example embodiments may be embodied in many different forms and that neither should be construed to limit the scope of the disclosure.

The terminology used herein is for the purpose of describing particular example aspects only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or feature is referred to as being “on,” “engaged to,” “connected to,” “coupled to” “operably connected to” or “in operable communication with” another element or feature, it may be directly on, engaged, connected or coupled to the other element or layer, or intervening elements or features may be present. In contrast, when an element is referred to as being “directly on,” “directly engaged to,” “directly connected to,” or “directly coupled to” another element or feature, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly and expressly indicated by the context. Thus, a first element, component, region, layer or section discussed below could be termed a second element, component, region, layer or section without departing from the teachings of the example embodiments.

For purposes of description herein, the terms “upper,” “lower,” “right,” “left,” “rear,” “front,” “vertical,” “horizontal,” and derivatives thereof shall relate to the invention as oriented in the FIGS. However, it is to be understood that the present disclosure may assume various alternative orientations and step sequences, except where expressly specified to the contrary. It is also to be understood that the specific devices and processes illustrated in the attached drawings, and described in the following specification are exemplary aspects of the inventive concepts defined in the appended claims. Hence, specific dimensions and other physical characteristics relating to the aspects disclosed herein are not to be considered as limiting, unless the claims expressly state otherwise.

As discussed above, at its most basic level diabetes is a disease of dysfunctional control of blood glucose levels. Type 1 diabetes is characterized by a lack of insulin production and type 2, at least initially, by a lack of sensitivity to the effects of insulin and often later in progression of the disease by a lack of insulin. Given these changes in insulin levels or sensitivity to insulin it is not surprising that since the 1920s the emphasis in virtually all forms of treatment of diabetes currently in use or being developed are directed toward insulin in one form or another. These include supplementation with insulin and methods to increase the effectiveness of insulin in the body. The present inventors have taken a different approach to diabetes treatment as discussed herein. Prior to discussion of the present invention some background discussion on the physiology of blood glucose control is important. First, a discussion of blood flow through the relevant organs and then a proposal of an alternative explanation of the disruption to blood glucose levels in diabetes and ways to correct the same.

Fully oxygenated blood leaves the heart via the aorta and enters the mesenteric arteries and other arteries to supply blood to the stomach, spleen, intestines and pancreas. Specifically, the celiac artery supplies blood to the stomach, spleen and the pancreas. The superior mesenteric artery supplies blood to the small intestine, large intestine, stomach and pancreas. The inferior mesenteric artery supplies blood flow to the transverse and descending colons and the rectum. The blood leaving the stomach, spleen, intestines and pancreas is collected in the hepatic portal system and all of these veins feed into the hepatic portal vein. The pancreas also receives blood from the splenic artery, the superior and inferior pancreaticoduodenal arteries. The hepatic portal vein provides 75% of the blood flow to the liver, the other 25% comes from the hepatic artery. The blood flow leaves the intestines via the mesenteric veins which eventually feed into the hepatic portal vein. The absorbed contents from the stomach, intestines and any endocrine or exocrine substances released into the blood flow from the pancreas are all collected in and concentrated in the hepatic portal vein and flow to the liver via the hepatic portal vein. The blood flows out of the liver via the central veins to the hepatic vein and then eventually returns to the heart via the inferior vena cava. Thus, the liver is bathed in concentrated levels of digested substances coming from the intestines like carbohydrates, proteins and fats and other nutrients from the digestion of food and both endocrine and/or exocrine substances from the pancreas. One of the main functions of the liver is to regulate metabolism and storage of nutrients, including glucose, from the intestine and stomach. Also the liver sees much higher levels of insulin, amylin and glucagon than the other cells in the body because of its location relative to the pancreas and because the majority of its blood flow is via the hepatic portal vein which is fed in part by the pancreatic veins.

The pancreas is a pear shaped organ having a head, neck, body and tail portion. The head portion is situated at the junction between the stomach and the start of the small intestine. The pancreas performs both an exocrine and an endocrine function, with 95% of the cells devoted to the exocrine function and only 5% to the endocrine function. The exocrine function has a major involvement in digestion as the exocrine glands release the main digestive enzymes from the head of the pancreas into a series of ducts that collect into the main pancreatic duct, which empties into the small intestine to aid in digestion. The endocrine function of the pancreas is performed by the islets of Langerhans cells which release hormones to regulate blood sugar levels and pancreatic secretions. The islets of Langerhans comprises two main types of cells alpha cells and beta cells, it also contains some delta cells. The alpha, beta and delta cells are in close proximity to each other in the islets of Langerhans. Thus, insulin and amylin release from beta cells is seen by alpha cells and glucagon release by alpha cells is seen by beta cells. Due to their close proximity to each other alpha and beta cells see very high levels of insulin, amylin and glucagon compared to other cells in the body. It is estimated that beta cells may see up to 100 times greater levels of insulin compared to other cells in the body. See Roger H. Unger and Alan D. Cherrington, Glucagonocentric restructuring of diabetes: a pathophysiologic and therapeutic makeover, The Journal of Clinical Investigation, Volume 122, Number 1, January 2012, pp 4-12. Thus insulin and amylin serve a paracrine function, within organ signaling, to reduce release of glucagon by alpha cells. These locally high levels of insulin and amylin are believed to be important for control of glucagon. The main pancreatic hormones are insulin, amylin and glucagon. Insulin and amylin are released by beta cells while glucagon is released by alpha cells. Insulin functions to lower blood sugar levels while glucagon raises blood sugar levels. Insulin and amylin as discussed also function to reduce glucagon release. The delta cells found in the islets of Langerhans release the hormone somatostatin. These delta cells are also found in other places in the body including in the pyloric antrum and the duodenum.

Insulin functions in the body as a “key” to unlock cells and allow glucose into the cells for fuel. Thus, following a meal digestion breaks down carbohydrates into glucose and other sugars. The sugars are absorbed into the blood stream and the elevation in blood glucose levels triggers release of insulin from the pancreas. Circulating insulin drives glucose into cells for use as fuel. The brain requires glucose as neurons cannot effectively use fats or proteins as fuel. Excess blood glucose is stored as glycogen by the liver and in skeletal muscle. As glucose levels fall in the blood release of glucagon by the pancreas causes the liver and skeletal muscle to break glycogen down into glucose through the process of glycogenolysis to maintain normal blood glucose levels. Glucagon also promotes lipolysis to breakdown stored triglycerides into fatty acids and gluconeogenesis to form glucose from amino acids. The promotion of lipolysis provides fatty acids for fuel use by cells other than neurons thereby saving the released glucose for use by the brain. During digestion of a high protein meal glucagon release promotes gluconeogenesis from the amino acids released by digestion of the proteins.

As discussed above, typical treatment for diabetes both type 1 and type 2, revolves around insulin supplementation or augmentation. The present inventors believe that there can be improvement in blood glucose control by turning more attention to regulation of glucagon rather than only insulin. They believe the proximal cause of elevated blood sugar and catabolism in type 1 diabetes isn't due to a lack of insulin directly, but rather due to an elevation of glucagon. This theory has been suggested by others also, see Roger H. Unger and Alan D. Cherrington, Glucagonocentric restructuring of diabetes: a pathophysiologic and therapeutic makeover; The Journal of Clinical Investigation; Volume 122, number 1; January 2012, pp 4-12. Glucagon signals the liver to release glucose via breakdown of glycogen and via gluconeogenesis from amino acids. It also signals lipolysis within adipose tissue and catabolism of glycogen and protein in muscle. In studies, using glucagon receptor null mice, meaning mice that are genetically altered so they do not produce glucagon receptors, these mice are shown to have well-controlled blood glucose levels and no symptoms of diabetes, both before and after destruction of their insulin-producing beta cells in the pancreas. By way of contrast the wild type mice which have intact and functional glucagon receptors have the opposite effect. The wild type mice have normal glucose control; however once the insulin-producing cells are destroyed in the pancreas they quickly develop type 1 diabetes. The level of circulating glucagon in these wild type mice increases significantly and within 6 weeks they needed to be sacrificed. The receptor null mice showed normal glucose levels and a normal response to a glucose tolerance test. They remained healthy showing no signs of type 1 diabetes for over 4 months following complete destruction of their insulin-producing beta cells. See Roger H. Unger and Lelio Orci, Paracrinology of islets and the paracrinopathy of diabetes; PNAS, Sep. 14, 2010; Vol. 107, no. 37; pp 16009-16012 and Young Lee, May-Yun Wang, Xiu Quan Du, Maureen J. Charron, and Roger H. Unger, Glucagon Receptor Knockout Prevents Insulin-Deficient Type 1 Diabetes in Mice, Diabetes Vol. 60, February 2011; pp 391-397. Even when the glucagon receptor null mice lack insulin production entirely, they do not suffer from uncontrolled blood sugar, sarcopenia, or ketoacidosis.

The present inventors propose a solution to treatment of diabetes comprising controlling glucagon levels rather than relying on subdermal injection of insulin alone or in combination with other insulin effect enhancing drugs. In a first embodiment, control of glucagon will be established through use of a drug eluting stent placed in one of the arteries or veins supplying blood to the pancreas. The stent will be designed to elute drugs that suppress the release of at least glucagon by alpha cells. Since the stent will be placed in the arterial or venous blood supply to the pancreas it can be assured that the pancreatic alpha cells will see higher levels of the glucagon suppressing drug than elsewhere in the body while still being able to keep the overall released amount of suppressing drug relatively low. The chosen glucagon suppressing drug will thus be targeted to the alpha cells of the pancreas. Candidates for the glucagon suppressing drugs according to the present disclosure include: somatostatin and commercial somatostatin analogues; leptin and commercial leptin analogues; amylin and commercial amylin analogues; insulin and commercial insulin analogues; and combinations of these glucagon suppressing drugs. It is believed that the use of a combination of glucagon suppressing drugs may result in a synergistic effect allowing for lower levels of each drug to be used compared to use of a single glucagon suppressing drug. It is believed that these proposed treatments will not only suppress glucagon release especially in type 1 diabetics but that they will also suppress excess insulin release by beta cells in type 2 diabetics.

Release of the hormone somatostatin by delta cells is triggered by the beta cell produced peptide Urocortin3 (Ucn3). It may be that in diabetics, especially type1 having no beta cells, that the absence of these beta cells in addition to effecting insulin production also reduces somatostatin release and thereby further increases the levels of glucagon in the diabetic patient. Somatostatin is released from a preproprotein in two forms due to alternative cleavage of the preproprotein. One form is 14 amino acids in length and the other is 28 amino acids in length. Somatostatin can effect neurotransmission, cell proliferation via interaction with G protein coupled somatostatin receptors and inhibition of the release of many secondary hormones including both insulin and glucagon. Somatostatin has clearly been shown to function to inhibit both insulin and glucagon release by the beta and alpha cells, respectively, of the pancreas. See Roger H. Unger and Alan D. Cherrington, Glucagonocentric restructuring of diabetes: a pathophysiologic and therapeutic makeover; The Journal of Clinical Investigation; Volume 122, number 1; January 2012, pp 4-12. Although somatostatin has other functions in the brain and digestive tract, a targeted dose at the right location using the inventive drug eluting stent is expected to serve to inhibit glucagon release without excessive effects elsewhere. Somatostatin analogues include, by way of example only, the octopeptide octreotide acetate (Sandostatin®) from Novartis. It is used to treat acromegaly, and for treatment of watery diarrhea, severe diarrhea and flushing episodes associated with vasoactive intestinal peptide (VIP) secreting tumors and metastatic carcinoid tumors. Another commercial version of somatostatin is lanreotide (Somatuline®) from Ipsen Pharmaceuticals. It is used for a similar treatment protocol.

Leptin is a hormone released from adipose tissue, fat cells. Its levels in the blood correlate with the total fat content in the body. Leptin has generally been studied for its effects on the feeding centers of the brain. Leptin regulates food intake and energy expenditure. Leptin can also regulate release of insulin and glucagon from the pancreas. See May-yun Wang, Lijun Chen, Gregory O. Clark, Young Lee, Robert D. Stevens, Olga R Ilkayeva, Brett R. Werner, James R. Bain, Maureen J. Charron, Christopher B. Newgard and Roger H. Unger, Leptin therapy in insulin-deficient type 1 diabetes, PNAS, Mar. 16, 2010, Vol. 107, No. 11, pp 4813-4819. Commercial leptin analogues include, by way of example, metreleptin.

Commercial analogues of amylin include, by way of example, Pramlinitide also known as Symlin, it was developed by Amylin Pharmaceuticals, which is now wholly owned by AstraZeneca. There are many commercial analogues of insulin as is known to those of skill in the art and thus they will not be listed here.

Drug eluting stents are currently used in cardiovascular recovery protocols, especially to release blood clot blocking drugs. The typical structure of a drug eluting stent comprises a metal mesh scaffolding formed from a biocompatible metal which is then covered with a biocompatible polymer. The drug to be eluted is typically placed into the polymer coating. In a typical example the drug either elutes out of the polymer or the polymer itself is biodegradable and the biodegradation releases the drug. Common metal mesh scaffolds comprise chromium in combination with cobalt, platinum or a combination thereof. Candidates for the polymers, eluting and biodegradable include: poly(L-lactic acid) (PLLA), also known as polylactide; polymers formed from amino acids such as tyrosine; poly(lactic-co-glycolic acid) (PLGA), a co-polymer of lactic acid and glycolic acid; polycaprolactone a biodegradable polyester polymer; poly(vinylidene fluoride-co-hexafluoropropylene) (PVDF-HFP); a mixture of poly(ethylene glycol) and poly(L-alanine-co-L-phenyl alanine) (PEG/PAF); or a block co-polymer of PEG-PCL-PEG which is formed from blocks of poly(ethylene glycol) and polycaprolactone; and combinations of these polymers. Typically, the glucagon suppressing drug will be mixed with or entrained into the polymer and then the mixture will be coated onto the metal mesh scaffolding material to form the drug eluting stent. In one example, a co-polymer of PLGA is dissolved in a solvent of benzyl benzoate and benzyl alcohol and the drug can be entrained in the polymer. The polymer is then precipitated out of the solvent, entraining drug with it, upon exposure to aqueous solutions to form a porous high surface area structure with the drug inside. The porosity can be tuned by controlling the precipitation conditions.

It is important to release a sufficient amount of glucagon suppressing drug from the stent without releasing an excess. This is important for the therapeutic benefits and to extend the time between replacement of the drug eluting stent once its release of glucagon suppressing drugs has ceased. It is estimated that under current insulin treatment methods for type 1 diabetics that the insulin usage per year is approximately 20,000 units of insulin at 0.8 units per kilogram (kg) per day and an average mass of 70 kg. A single unit of insulin equals 6 nanomoles (nM) or 0.035 milligrams (mg). Per animal studies an insulin concentration of 300 picomoles/Liter, 1.7 micrograms/Liter, was sufficient to suppress glucagon release by alpha cells. See Elisa Vergari, Jakob G. Knudsen, Reshma Ramrecheya, Albert Salehi, Quan Zhang, Julie Adam, Ingrid Wernstedt Asterholm, Anna Berick, Linford J. B. Briant, Margarita V. Chibalina, Fiona M. Gribble, Alexander Hamilton, Benoit Hastoy, Frank Reimann, Nils J. G. Rorsman, loannis I. Spiliotis, Andrei Tarasov, Yanling Wu, Frances M. Ashcroft and Patrik Rorsman, Insulin inhibits glucagon release by SGLT2-induced stimulation of somatostatin secretion, Nature Communications, (2019) 10:139, pp 1-11. The estimated blood flow to the pancreas is up to 1 milliliter/minute per gram. See Leif Jansson, Andreea Barbu, Brigitta Bodin, Carl Johan Drott, Daniel Espes, Xiang Gao, Liza Grapensparr, Orjan Kallskog, Joey Lau, Hanna Liljebäck, Fredrik Palm, My Quach, Monica Sandberg, Victoria Stromberg, Sara Ullsten and Per-Ola Carlsson, Pancreatic islet blood flow and its measurement, Upsala Journal of Medical Sciences, 2016, VOL. 121, NO. 2, 81-95. The average pancreas weight is approximately 80 grams, so blood flow through it is approximately 80 milliliters/minute. The total blood flow in the body of an adult is approximately 5,000 milliliters/minute, so the pancreas blood flow represents 1.6% of the total blood flow. The average blood volume for a human is approximately 5 liters. Using these values it is estimated that continuous suppression of glucagon release by insulin would require approximately 71 milligrams of insulin per year which is equivalent to 2000 units per year. This is far below the average usage of 20,000 units per year under current treatment protocols. Similar calculations can be undertaken for the amount of other glucagon suppressors such as somatostatin and its commercial analogues, leptin and its commercial analogues and amylin and its commercial analogues. It has been reported that intravenous infusion of 25 micrograms per hour of Pramlintide, an amylin analogue, could suppress a glucagon spike from a standardized meal in a Type 1 diabetic. See M. S. Fineman, J. E. Koda, L. Z. Shen, S. A. Strobel, D. G. Maggs, C. Weyler, and O. G. Kolterman, The Human Amylin Analog, Pramlintide, Corrects Postprandial Hyperglucagonemia in Patients With Type 1 Diabetes, Metabolism, Vol 51, No 5, 2002, pp 636-641. Based on the data in this report and the half life of pramlintide one can estimate a yearly requirement of approximately 303 milligrams per year required for suppression of glucagon spikes. It has been reported that in an in vitro systems of human alpha-cells a concentration of 0.625 nanomoles/Liter of leptin could suppress their functional response to glucose. See Eva Tuduri, Laura Marroqui, Sergi Soriano, Ana B. Ropero, Thiago M. Botista, Sandra Piquer, Miguel A. Lopez-Boado, Everado M. Carneiro, Ramon Gomis, Angel Nadal and Ivan Quesada, Inhibitory Effects of Leptin on Pancreatic α-Cell Function, Diabetes, Vol. 58, July 2009, pp 1616-1624. Using this data one can calculate a yearly requirement of 421 milligrams per year. Finally, in a report it was shown that an intravenous infusion of 500 micrograms per hour of somatostatin suppressed glucagon spikes in Type 1 diabetics. Using its half life of 3 minutes one can calculate a requirement for 295 milligrams per year to suppress glucagon. See John E. Gerich, M. D., Mara Lorenzi, M. D., Dennis M. Bier, M. D., Victor Schneider, M. D., Evan Tsalikian, M. D., John H. Karam, M. D., and Peter H. Forsham, M. D., Prevention of Human Diabetic Ketoacidosis by Somatostatin Evidence for an Essential Role of Glucagon, The New England Journal of Medicine, Volume 292, May 8, 1975, Number 19, pp 985-989. These calculations are generalizations and one can expect the therapeutic window to be influenced by the efficacy of the glucagon suppressing drug, its half life in the body, bioavailability, and partitioning among other factors. In general, the rate of elution from the stent can be estimated to range from 50 to 500 mg per year depending on the compound used. Understanding that in diabetes, excessive glucagon secretion by the alpha cells amounts to an ambient hyperglucagonemia of approximately 25-50% above levels of plasma glucagon observed in non-diabetics, one can anticipate an targeted suppression of glucagon in the range of 10 to 60% from the pre-treatment levels would result in meaningful metabolic benefits in the diabetic patient. The composition of the stent polymer and how the drugs are entrained in the polymer will influence the rate of release. The release rate must be sufficient to suppress the excess glucagon release seen in type 1 and type 2 diabetics, thereby restoring glucose homeostasis.

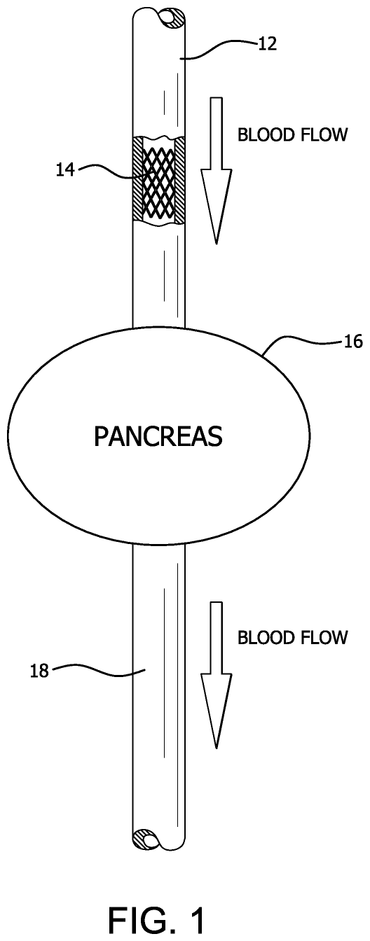

FIG. 1 shows a schematic diagram illustrating the first embodiment of the present disclosure. FIG. 1 shows an arterial or venous blood vessel 12 feeding into the pancreas 16 and blood flowing out of the pancreas 16 through the hepatic portal vein 18. A drug eluting stent 14 is implanted into one of the vessels 12 feeding the pancreas 16. The drug elutes from the stent 14 and into the pancreas 16 with the blood flow. Thus, exposing the pancreas 16 to the eluted drug as it is eluted from the stent 14. This will provide high levels of the drug to the alpha and beta cells of the pancreas 16, leading to suppression of the release of glucagon from the alpha cells.

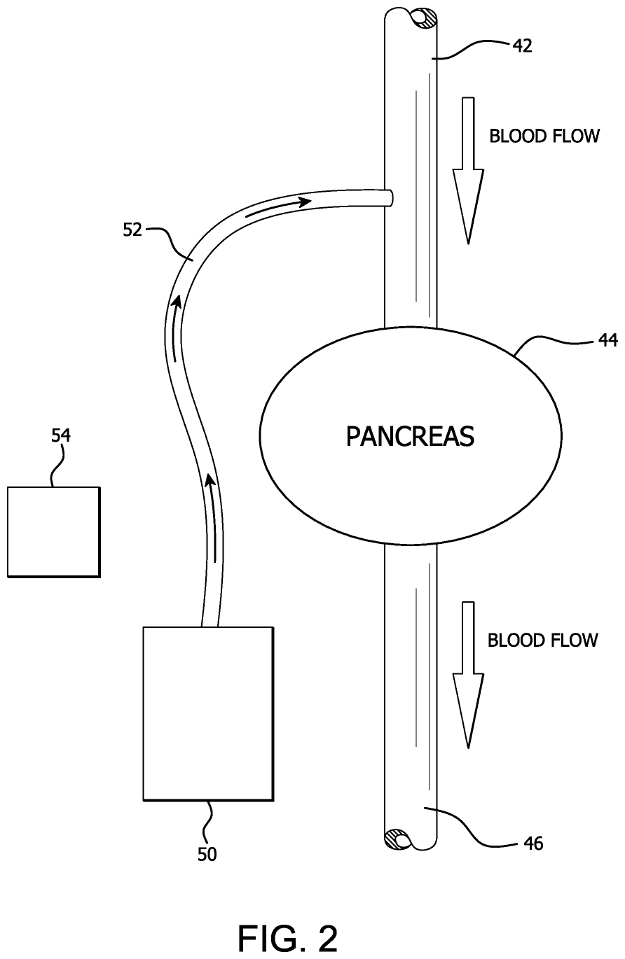

A second embodiment of the present disclosure is shown schematically in FIG. 2. As shown an artery 42 supplies blood flow to the pancreas 44 and the blood flows through the pancreas 44 and out of the hepatic portal vein 46. A pump 50 is shown, the pump 50 includes at least one reservoir, not shown, containing at least one glucagon suppressing drug. The pump 50 includes a catheter 52 going from the pump 50 and into the artery 42 supplying the pancreas 44. The system optionally includes a continuous glucose monitor sensor 54 which interfaces with the pump 50 as known in the art to communicate interstitial blood glucose levels to the pump 50. The catheter 52 is inserted into an artery 42 feeding the pancreas 44. The pump 50 is programmable and adjustable as is known in the art for current insulin pump systems. The pump 50 is programmed to deliver one or more glucagon suppressing drugs from its reservoir, not shown. The rate of delivery from the pump 50 can be varied over time as determined by the user, generally in conjunction with their endocrinologist. The rate of delivery can be altered as needed and a bolus of the glucagon suppression drug can be delivered at a mealtime. The glucagon suppression drugs are as described above and include somatostatin, somatostatin analogues, amylin, amylin analogues, leptin, leptin analogues, insulin, insulin analogues, and combinations of any of these drugs. It is believed that use of a combination of glucagon suppressing drugs may result in a synergistic effect such that less of each drug can be used to achieve the same effect from use of a single glucagon suppressing drug. It is anticipated that given the point of entry, an artery supplying the pancreas, that like the stent embodiment the system will provide high levels of the glucagon suppression drugs to the alpha and beta cells of the pancreas while keeping systemic levels relatively low. When the optional continuous glucose monitor sensor 54 is used the readings of interstitial glucose that it sends to the pump can be used to adjust the rate of flow of the glucagon suppression drugs as need to maintain glucose homeostasis. This is similar to current insulin pumps which vary their output of insulin in response to signals from the continuous glucose monitor sensor. In a further refinement of this embodiment it is anticipated that the pump and reservoir system can be reduced in size sufficiently to allow for it to be implanted internally in the patient. In such an example the reservoir can include a self-sealing membrane to allow for refilling of the reservoir as is found in other implantable drug delivery devices. The battery of the implantable pump can be rechargeable by wireless magnetic induction as is known for other implantable pumps.

Summarising, this disclosure may be considered to relate to the following items:
1. A stent comprising a metal mesh scaffolding, said stent comprising a biocompatible polymer coating and said biocompatible polymer coating containing at least one glucagon suppressing drug wherein said drug elutes from said biocompatible polymer coating over time.
2. The stent of item 1, wherein said metal mesh scaffolding comprises chromium in combination with cobalt, platinum or a combination thereof.
3. The stent of item 1 or 2, wherein said biocompatible polymer coating comprises poly(L-lactic acid); a polymer comprising one or more amino acids; poly(lactic-co-glycolic acid); polycaprolactone; poly(vinylidene fluoride-co-hexafluoropropylene); a poly(ethylene glycol) poly(L-alanine-co-L-phenyl alanine) co-polymer; block co-polymers of poly(ethylene glycol) and poly(caprolactone); or combinations thereof.
4. The stent of any of the foregoing items, wherein said at least one glucagon suppressing drug comprises somatostatin, a somatostatin analogue, leptin, a leptin analogue, amylin, an amylin analogue, insulin, and insulin analogue, or combinations thereof.
5. The stent of any of the foregoing items, wherein said at least one glucagon suppressing drug elutes from said biocompatible polymer coating at a rate of from 50 to 500 milligrams per year.
6. A method of treating diabetes comprising the following steps:
a) providing a stent comprising a metal mesh scaffolding, stent comprising a biocompatible polymer coating and the biocompatible polymer coating containing at least one glucagon suppressing drug wherein the drug can elute from the biocompatible polymer coating over time;
b) identifying a patient having diabetes;
c) inserting the stent into an artery or a vein supplying blood to the pancreas of the identified patient, thereby treating the diabetes.
7. The method of item 6, wherein step a) further comprises providing a metal mesh scaffolding comprising chromium in combination with cobalt, platinum or a combination thereof.
8. The method of item 6 or 7, wherein step a) further comprises providing a biocompatible polymer coating comprising poly(L-lactic acid); a polymer comprising one or more amino acids; poly(lactic-co-glycolic acid); polycaprolactone; poly(vinylidene fluoride-co-hexafluoropropylene); a poly(ethylene glycol) poly(L-alanine-co-L-phenyl alanine) co-polymer; block co-polymers of poly(ethylene glycol) and poly(caprolactone); or combinations thereof.
9. The method of any of items 6 to 8, wherein step a) further comprises the biocompatible polymer coating containing at least one glucagon suppressing drug comprising somatostatin, a somatostatin analogue, leptin, a leptin analogue, amylin, an amylin analogue, insulin, and insulin analogue, or combinations thereof.
10. The method of any of items 6 to 9, wherein step a) further comprises providing a stent wherein the at least one glucagon suppressing drug elutes from the biocompatible polymer coating at a rate of from 50 to 500 milligrams per year.
11. The method of any of items 6 to 10, wherein step c) comprises inserting the stent into one of the celiac artery, the superior mesenteric artery, the inferior mesenteric artery, the splenic artery, the superior pancreaticoduodenal artery, the inferior pancreaticoduodenal artery, or a vein supplying blood to the pancreas.
12. A method of treating diabetes comprising the following steps:
   a) providing a pump having a catheter and at least one reservoir containing at least one glucagon suppressing drug;
   b) identifying a patient having diabetes;
   c) inserting the catheter into an artery supplying blood to the pancreas; and
   d) infusing the at least one glucagon suppressing drug into the artery from the catheter, thereby treating the diabetes.
13. The method of item 12, wherein step a) further comprises providing as the at least one glucagon suppressing drug somatostatin, a somatostatin analogue, leptin, a leptin analogue, amylin, an amylin analogue, insulin, and insulin analogue, or combinations thereof.
14. The method of item 12 or 13, wherein step c) comprises inserting the catheter into one of the celiac artery, the superior mesenteric artery, the inferior mesenteric artery, the splenic artery, the superior pancreaticoduodenal artery, or the inferior pancreaticoduodenal artery.
15. The method of any of items 12 to 14, further comprising providing a continuous glucose monitor sensor, the continuous glucose monitor sensor measuring interstitial glucose levels and communicating the same to the pump.
16. The method of item 15, wherein the pump adjusts a rate of infusion of the at least one glucagon suppressing drug based on the measured interstitial glucose level.
17. The method of any of items 12 to 16, further comprising the step of implanting the pump into the identified patient.

Any of the embodiments and/or elements disclosed herein may be combined with one another to form various additional embodiments not specifically disclosed, as long as they do not contradict each other. It is particularly noted that those skilled in the art can readily combine the various technical aspects of the various elements of the various exemplary embodiments that have been described above in numerous other ways, all of which are considered to be within the scope of the invention, which is defined by the appended claims and their equivalents.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure. Accordingly, the scope of legal protection afforded this disclosure can only be determined by studying the following claims.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

1. A stent comprising a metal mesh scaffolding, said stent comprising a biocompatible polymer coating and said biocompatible polymer coating containing at least one glucagon suppressing drug wherein said drug elutes from said biocompatible polymer coating over time.

2. The stent according to claim 1, wherein said metal mesh scaffolding comprises chromium in combination with cobalt, platinum or a combination thereof.

3. The stent according to claim 1, wherein said biocompatible polymer coating comprises poly(L-lactic acid); a polymer comprising one or more amino acids; poly(lactic-co-glycolic acid); polycaprolactone; poly(vinylidene fluoride-co-hexafluoropropylene); a poly(ethylene glycol) poly(L-alanine-co-L-phenyl alanine) co-polymer; block co-polymers of poly(ethylene glycol) and poly(caprolactone); or combinations thereof.

4. The stent according to claim 1, wherein said at least one glucagon suppressing drug comprises somatostatin, a somatostatin analogue, leptin, a leptin analogue, amylin, an amylin analogue, insulin, and insulin analogue, or combinations thereof.

5. The stent according to claim 1, wherein said at least one glucagon suppressing drug elutes from said biocompatible polymer coating at a rate of from 50 to 500 milligrams per year.

6. A method of treating diabetes comprising the following steps:
a) providing a stent comprising a metal mesh scaffolding, stent comprising a biocompatible polymer coating and the biocompatible polymer coating containing at least one glucagon suppressing drug wherein the drug can elute from the biocompatible polymer coating over time;
b) identifying a patient having diabetes;
c) inserting the stent into an artery or a vein supplying blood to the pancreas of the identified patient, thereby treating the diabetes.

7. The method according to claim 6, wherein step a) further comprises providing a metal mesh scaffolding comprising chromium in combination with cobalt, platinum or a combination thereof.

8. The method according to claim 6, wherein step a) further comprises providing a biocompatible polymer coating comprising poly(L-lactic acid); a polymer comprising one or more amino acids; poly(lactic-co-glycolic acid); polycaprolactone; poly(vinylidene fluoride-co-hexafluoropropylene); a poly(ethylene glycol) poly(L-alanine-co-L-phenyl alanine) co-polymer; block co-polymers of poly(ethylene glycol) and poly(caprolactone); or combinations thereof.

9. The method according to claim 6, wherein step a) further comprises the biocompatible polymer coating containing at least one glucagon suppressing drug comprising somatostatin, a somatostatin analogue, leptin, a leptin analogue, amylin, an amylin analogue, insulin, and insulin analogue, or combinations thereof.

10. The method according to claim 6, wherein step a) further comprises providing a stent wherein the at least one glucagon suppressing drug elutes from the biocompatible polymer coating at a rate of from 50 to 500 milligrams per year.

11. The method according to claim 6, wherein step c) comprises inserting the stent into one of the celiac artery, the superior mesenteric artery, the inferior mesenteric artery, the splenic artery, the superior pancreaticoduodenal artery, the inferior pancreaticoduodenal artery, or a vein supplying blood to the pancreas.

12. A method of treating diabetes comprising the following steps:
a) providing a pump having a catheter and at least one reservoir containing at least one glucagon suppressing drug;
b) identifying a patient having diabetes;
c) inserting the catheter into an artery supplying blood to the pancreas; and
d) infusing the at least one glucagon suppressing drug into the artery from the catheter, thereby treating the diabetes.

13. The method according to claim 12, wherein step a) further comprises providing as the at least one glucagon suppressing drug somatostatin, a somatostatin analogue, leptin, a leptin analogue, amylin, an amylin analogue, insulin, and insulin analogue, or combinations thereof.

14. The method according to claim 12, wherein step c) comprises inserting the catheter into one of the celiac artery, the superior mesenteric artery, the inferior mesenteric artery, the splenic artery, the superior pancreaticoduodenal artery, or the inferior pancreaticoduodenal artery.

15. The method according to claim 12, further comprising providing a continuous glucose monitor sensor, the continuous glucose monitor sensor measuring interstitial glucose levels and communicating the same to the pump.

16. The method according to claim 15, wherein the pump adjusts a rate of infusion of the at least one glucagon suppressing drug based on the measured interstitial glucose level.

17. The method according to claim 12, further comprising the step of implanting the pump into the identified patient.
